(19) United States
(12) Patent Application Publication
(10) Pub. No.: US 2020/0129574 A1
(43) Pub. Date: Apr. 30, 2020

(54) AMARANTHUS BLITOIDES WITH VITAMIN D COMPOSITIONS TO ENHANCE VITAMIN D HALF-LIFE AND ACTIVATE VDR IN HUMANS AND METHODS OF USING SAME

(71) Applicant: David Audino, Park Ridge, IL (US)

(72) Inventor: David Audino, Park Ridge, IL (US)

(21) Appl. No.: 16/661,069

(22) Filed: Oct. 23, 2019

Related U.S. Application Data
(60) Provisional application No. 62/749,976, filed on Oct. 24, 2018.

(51) Int. Cl. A61K 36/21; A61K 31/593; A61P 29/00; A23K 10/30; A23L 33/105; A23L 2/52; A23L 33/155; A23K 20/174; A23F 3/34; A23P 10/30

(52) U.S. Cl. CPC A61K 36/21; A61K 31/593; A61P 29/00; A23K 10/30; A23L 33/105; A23V 2002/00; A23L 33/155; A23K 20/174; A23F 3/34; A23P 10/30; A23L 2/52

(57) ABSTRACT

The present invention discloses a composition, which includes Amaranthus blitoides and Vitamin D or its derivatives. The present invention also provides methods for treating inflammatory conditions in humans or animals by administering the compositions presently claimed. The composition of the invention can effectively alleviate inflammation and can treat benign prostatitis, and has good application prospect.

### BACKGROUND OF THE INVENTION

This application claims the benefit of U.S. Provisional Application No. 62/749,976, filed on Oct. 24, 2018 entitled “Amaranthus blitoides with Vitamin D compositions to enhance Vitamin D half life and activate VDR in humans and methods of using same.” The present invention is related to U.S. application Ser. No. 15/220,846, filed on Jul. 27, 2016, entitled “Enriched Amaranthus Blitiodes Compositions and Uses Thereof.”

Amaranthus blitoides is a common member of the pigweed family. It occurs in much of the United States and Canada, as well as in Europe and other areas of the world.

Amaranthus blitoides. Amaranthus blitoides is an edible herb of the family Amaranthaceae. It is often referred to as Prostrate amaranth, Matweed, and Mat. It is has been used as a food for both humans and domestic animals for centuries and no significant side effects have been observed during this period. It is particularly popular in many countries in Europe including, for instance, Germany, France, Italy, Croatia, Serbia and Montenegro, and it is commonly available in various forms in health food and organic food shops in the United States. It is made into flour and used to make breads, pastas and other baked goods. There is no gluten in Amaranthus.

It is, like all the Amaranthaceae, monoecious. It has separate male (staminate) and female flowers (pistillate). Male flowers have 4-5 sepals, 3 stamens, and no petals. Female flowers have 4-5 sepals, an ovary with 3 styles, and no petals. The sepals of both male and female flowers are oblong lanceolate shaped and approximately ⅛″ in length.

It is believed to have originated in central or eastern United States. Today, it is found in the wild throughout much of temperate North America, South America and Eurasia. Typically, it grows in moist fertile soil in sunny areas. Plants generally grow to 0.6 m to 1 m. They are summer annuals and wind pollinated. Flowers have three stigmas in the form of axillary clusters. The cuspidate floral bracts are as long as the flowers themselves. Each flower gives rise to a capsule. Each capsule releases one seed. The seeds are edible round, flattened discs, shiny dark brown to black in appearance, about 1.1-1.7 mm in diameter. The plant and its cultivation, harvesting, drying and preparation are well known, and it is available commercially in several forms.

Amaranthus blitoides , on a weight per cent basis, contains more protein than milk, three times as much fiber and five times as much iron as wheat, and high levels of vitamins A and C, calcium, potassium, and phosphorous. The seeds contain fairly high levels of lysine, which is lacking in other grains. The plant also contains many macro and micro nutrients including, but not limited to vitamin C, carotene, starch, proteins, fatty oils, sugar, cellulose, flavonoids, tannins, betanine, saponins, glycoproteins, 13-sitosterol, TRF, Fe, Mn, Cu, Co, K, Zn, and Na, and it is a rich source of camesterol and stigmasterol. It has long been known for its analgesic, antiphologistic, and spasmolytic properties, no doubt owing to these and other ingrediants. For instance, flavonoids bind free radicals and have anti-inflammatory, antimicrobial, and antiviral effects. Saponins have been shown to have anti-inflammatory effects. The proteins and glycoproteins in the plant are reputed to have antimicrobial and antiedematous effects. B-sitosterol is a phytosterol that may exercise anti-inflammatory effects by interfering with prostaglandin metabolism, and it has been reported to have anti-benign prostatic hyperplasia (BPH) activity. Two other phytosterols found in Amaranthus, camesterol and stigmasterol, have been reported to improve cholesterol levels and to be of some help to those suffering from peripheral artery and coronary artery disease.

Amaranthus blitoides has been used for some time, particularly as tea, for relief from a variety of discomforts and symptoms, such as those caused by or associated with inflammation and inflammatory processes, such as symptoms of Benign Prostatic Hyperplasia (BPH), and those often associated with infections of the genitourinary tract or hemorrhoidal diseases.

Various preparations of A. blitoides, especially primarily preparations for making teas, are available commercially in Europe,. Its widespread use in Europe, particularly for relief from BPH, is popular confirmation that it is effective in relieving discomfort; something that has been confirmed by more systematic studies. For example, The book of Bogdan Boskovic, “Pharmacological Toxicological-Clinical Expert Report for Amer Tea”; National Center for Toxic, Belgrade, Serbia (2005) has recorded the effectiveness of A. blitoides, which is herein incorporated in its entirety in parts pertinent to the use of Amaranthus to treat disorder or disease, or use for other medicinal purposes.

Vitamin D. Vitamin D is an essential nutrient in human physiology.The two forms primarily of importance to human nutrition are Vitamin D2 (ergocalciferol) and Vitamin D3 (cholecalciferol). Other forms of vitamin D include Vitamin D1 (a 1:1 mixture of ergocalciferol and lumisterol), Vitamin D4 (22-dihydroergocalciferol) and Vitamin D5 (sitocalciferol). Vitamins D2 and D3 both are precursors to physiologically active compounds. Both are activated by two hydroxyl addition reactions. The first generally occurs in the liver. The second occurs either in kidney, resulting in hormone activity, or in cells of the immune system, in which case the activated vitamin exhibits cytokine activity.

The activated form of Vitamin D binds to vitamin D receptors. The Vitamin D-Vitamin D Receptor complexes (vD-vDR) form heterodimers with retinoid X receptors (RXR). The heterodimers bind to vitamin D-responsive elements in the regulatory regions of vitamin D sensitive genes. Binding of the heterodimers to the genes effectuates changes in transcription. The transcriptional effects are mediated not only by heterodimer binding but also by a panoply of co-activators, including CBP/SRC coactivator complex, Vitamin D receptor interacting protein complex(DRIP), RAS activated Ets transcription factor, SWI/SNF complex and members of the CCAAT enhancer binding protein family. Vitamin D-responsive genes generally encode proteins involved in calcium and phosphate transport and its regulation, particularly transport proteins, of which TRPV6 and calbindin are particularly notable.

The molecular biology minors the main physiological activity of Vitamin D in regulating proper levels of calcium and phosphorous, particularly through their absorption in the intestine. These activities explain well known pathophysiologiocal effect of Vitamin D deficiency: inadequate mineralization and—via mobilization of calcium, in particular from bone—demineralization, causing rickets in children and ostomalacia and hyperparathyroidism in adults.

Vitamin D deficiencies are largely prevented in the economically developed countries of the world by government mandated supplementation of certain foods with Vitamin D. In the US, for instance, this is accomplished mainly by “fortifying” milk with 100 IU of Vitamin D per cup (approximately 2.5 microgram of Vitamin D per cup of milk). However, there is evidence suggesting that many Americans, particularly women, still suffer from Vitamin D deficiency and that it would be very beneficial for them to have additional Vitamin D in their diets. See Christakos et al. (2003), J. Cellular Biochem. 88: 695-705; Christakos et al. (2006), Ann. N.Y Acad. Sci. 1068: 194-203; Christakos et al. (2007), Ann, N.Y. Acad. Sci. 1116: 340-348 and National Report on Biochemical Indicators of Diet and Nutrition in the U.S. Population (1999-2002), Fat Soluble Vitamins & Nutrients: Vitamin D; pgs 61-69, for example). Moreover, there are populations that do not tolerate milk and would benefit from an additional supplemented dietary source. Vitamin D enriched Amaranthus blitoides provides such an alternative source of the Vitamin D.

Vitamin D also has been shown to play a role in immune regulation and to have activity against certain types of cancer. It has been shown to inhibit proliferation and promote differentiation of several tumor cell types, including prostate cancer cell lines, and primary prostate tumor lines, and to decrease invasion of prostate cancer cells in vitro (Sung et al. (200x): 1,25-Dihydroxyvitamin D3 decreases human prostate cancer cell adhesion and migration” Department of medicine, Division of Endocrinology, Gerontology and Metabolism, Stanford U., Sch. Med, Web). In addition, Vitamin D has been shown to induce decreased expression of a6 and 134 integrins, which are associated with increased migration and invasion of prostate cancer cells in vitro (Sung et al.(2000) as above and Sung et al. (2000), Malec. Cellular Endocrin. 164: 133-143. Furthermore, the Vitamin D analog, BXL-628 has been shown to arrest prostate growth and reduce prostate volume in Phase Ila clinical trials (Colli et al. (2006), European Urology 49: 82-86). See also WO2005/027923: Use of Vitamin D3 Analogs to Treat Prostate Hyperplasia.

Sources of Vitamin D Compounds. Vitamin D is available commercially in a variety of forms suitable for human consumption. In this regard it may be obtained as any one or a mixture of any combination of Vitamin D vitamers including Vitamin D1 , Vitamin D2, Vitamin D3, Vitamin D4 , and/or Vitamin D5 .

Vitamin D3, for example, is available commercially in a variety of soluble formulations suitable for human consumption. Falcon Trading International, offers Vitamin D3 Chlecalciferol; 40,300,000 IU/gram by HPLC, Grade USP25-BP2000, soluble white crystallinepowder, Melting Point 82-87 ”C; Absorption 0.46-0.50; Optical Rotation +105-+112°; Specific activity 38,800,000-41,200,000 IU/g (97.0-103.0% purity); 7-Dehydrocholesterol<0.2%.LycoRed, another company, provides a variety of Vitamin D formulations suitable for human consumption. One formulation is a powdered form in which the vitamin D is stabilized in a beadlet matrix suitable for use in dietary supplements, and for tablet and hard-shell capsule applications. Vitamin D2 and D3 formulations of this type are offered, in the following specific activities: Vitamin D2 100,000 and 850,000 IU/g; Vitamin D3 100,000, 500,000 and 1,000,000 IU/g. Microencapsulated powders specially formulated for food and beverage fortification also are available, including Vitamin D2 CapsuDar® D2 100E (100,000 IU/g) and Vitamin D3 CapsuDar® D3 1OOE (100,000 IU/g). Cold water dispersible powder formulations suitable for food and beverage fortification also are available, including Vitamin D2 CapsuDar® D2 100 CWD (100,000 IU/g) and Vitamin D3 CapsuDar® D3 100 CWD (100,000 IU/g). And oil suspensions suitable for use in soft-gel capsules and oil based applications are available from this company, including formulations of Vitamin D2 at 1,000,000 IU/g and Vitamin D3 at 1,000,000 IU/g.

Metabolism and Dosage of Vitamin D. Healthy adults tolerate a wide range of Vitamin D doses; but, it can be toxic in excessive amounts. Toxicity has been seen in healthy adults at dosages above about 1250 μg/day. Daily ingestion of 375 μg/kg (about 15 mg for a 90 lb person) of Vitamin D/day has been shown to produce acute toxicity in adults within two weeks. Toxicity has been observed in adults following long term daily ingestion of 25 μg/kg of Vitamin D (about 1 mg for a 90 lb person). Certain medical conditions increase sensitivity to vitamin D and, in extreme cases, even mildly elevated Vitamin D levels may result in adult hypercalcaemia. Hypercalcaemia in pregnant women has been associated with congenital mental retardation and facial deformities. See, for instance, Remington: The Science and Practice of Pharmacy 21st Ed., P. Beringer, pps 1696-1697, Lippincott, Williams & Wilkins, Baltimore, Md. (2006).

Vitamin D3 was found for the first time in Amaranthus blitoides . After testing, the content of vitamin D3 in Amaranthus blitoides is less than 0.020 IU/g, and the content of vitamin D2 is less than 0.0800 IU/g.

The palliative effect of preparations of A. Blitoides is incomplete, as is their curative power. All too often their use does not relieve symptoms and discomfort or underlying disease as fully as users and careproviders desire. In order to solve the above problems, we provide a novel composition including Amaranthus blitoides , which in order to get more effective teas and other administrative forms for the relief of discomforts and symptoms, such as those due to or associated with inflammation, for example symptoms commonly due to Benign Prostatic Hyperplasia (BPH), prostititous, including prostititous of the female prostate, infections of the genitourinary tract, and hemorrhoidal diseases, and for treatment of these diseases as well.

### SUMMARY

The present invention provides a composition having enhanced Vitamin D half life comprising Amaranthus blitoides and Vitamin D or its derivatives.

In an embodiment, the half life of Vitamin D or its derivatives is extended.

In any of the foregoing embodiments, the Vitamin D or its derivatives is substantially one or more of Vitamin D1 , Vitamin D2, Vitamin D3, Vitamin D4, Vitamin D5, or one or more derivatives thereof.

In any of the foregoing embodiments, the Amaranthus blitoides is Amaranthus blitoides Watson.

In any of the foregoing embodiments, the Amaranthus blitoides comprises dried leaves of Amaranthus blitoides Watson.

In any of the foregoing embodiments, the dried leaves comprise whole leaves, fragments, powder, or a mixture of whole leaves, fragments, and powder.

In any of the foregoing embodiments, the ratio of Amaranthus blitoides to Vitamin D or its derivatives is 1 g to 1, 2, 3, 4, 5, 10, 15, 20, 25, 30, 40, 50, 60, 75, 100, 125, 150, 175, 200, 225, 250, 275, 300, 325 or 350 IU. In another embodiment, the ratio of Amaranthus blitoides to Vitamin D or its derivatives is 1 g to 1-2000 IU, 2 g to 100 IU, or 5 g to 400 IU.

In any of the foregoing embodiments, the composition comprises minerals, organics, vitamins, amino acids, or other nutrients suitable for human consumption.

In any of the foregoing embodiments, the composition comprises any one or more of the following: zinc, copper, iodine, chromium, iron, molybdenum, selenium, silicon, boron, vanadium, germanium, potassium, calcium and/or magnesium; polyphenolics, sterols, such as sitosterol, and/or free fatty acids; Vitamin C, Vitamin E, Vitamin B-6 and/or other vitamins; lysine, glycine and/or tyrosine; saw palmetto, lycopene, quercetin, Pyseum africanum, stinging netttles, pumpkin seed oil, sasparilla, ginseng, Bubella broom, milk thistle, kelp, Uva ursi extract, flower pollen, bosvellio, flaxseed, bees wax, calcium D glycan, and/or glycinine.

In any of the foregoing embodiments, the composition comprises animal feed.

In any of the foregoing embodiments, the composition comprises a food and one or more pharmaceutically acceptable excipients or auxiliary constituents.

In any of the foregoing embodiments, the food comprises candy, bread, cake, pastry, or biscuits.

In another embodiment, the food comprises a liquid. In an embodiment, the composition is placed in a water permeable container. In any of the foregoing embodiments, the water permeable container comprises filter paper, nylon, silk or soilon, or tea bags. In any of the foregoing embodiments, the ratio of Amaranthus blitoides to Vitamin D is 2 g to 100 IU or 5 g to 400 IU. In any of the foregoing embodiments, wherein in one water permeable container, the weight of Amaranthus blitoides is 0.1 to 500 g. In any of the foregoing embodiments, the liquid comprises an energy drink, tea, coffee, lemonade, juice, juice drink, soft drink, water, flavored water drink, or nutritional food drink.

In any of the foregoing embodiments, the food is tea, tablet, capsule, oral liquid, pulvis, decoction, granule, or pill. In any of the foregoing embodiments, the ratio of Amaranthus blitoides to Vitamin D is 0.6718 g to 400 IU. In any of the foregoing embodiments, the weight of Amaranthus blitoides comprises 0.6718 g, and the Vitamin D comprises 400 IU. In any of the foregoing embodiments, the weight of Amaranthus blitoides is 0.1 to 500 g.

The present invention also provides a composition having enhanced Vitamin D half life comprising Amaranthus blitoides and Vitamin D or its derivatives, wherein the half life of Vitamin D or its derivatives is extended, wherein the Vitamin D or its derivatives is substantially one or more of Vitamin D1, Vitamin D2, Vitamin D3, Vitamin D4, Vitamin D5 or one or more derivatives thereof, wherein the Amaranthus blitoides is Amaranthus blitoides Watson, and wherein the ratio of Amaranthus blitoides to Vitamin D or its derivatives is 1 g to 1-2000 IU, 2 g to 100 IU, or 5 g to 400 IU.

In an embodiment, the composition comprises any one or more of the following: zinc, copper, iodine, chromium, iron, molybdenum, selenium, silicon, boron, vanadium, germanium, potassium, calcium and/or magnesium; polyphenolics, sterols, such as sitosterol, and/or free fatty acids; Vitamin C, Vitamin E, Vitamin B-6 and/or other vitamins; lysine, glycine and/or tyrosine; saw palmetto, lycopene, quercetin, Pyseum africanum, stinging netttles, pumpkin seed oil, sasparilla, ginseng, Bubella broom, milk thistle, kelp, Uva ursi extract, flower pollen, bosvellio, flaxseed, bees wax, calcium D glycan, and/or glycinine.

In any of the foregoing embodiments, the composition comprises a food and one or more pharmaceutically acceptable excipients or auxiliary constituents.

In any of the foregoing embodiments, the food comprises a liquid, wherein the composition is placed in a water permeable container, and wherein in one water permeable container the weight of Amaranthus blitoides is 0.1 to 500 g.

In any of the foregoing embodiments, the food comprises tea, tablet, capsule, oral liquid, pulvis, decoction, granule, or pill, and wherein the weight of Amaranthus blitoides is 0.1 to 500.

The present invention also provides a method for treating inflammation in humans or animals, the method comprising administering the presently claimed compositions.

The present invention also provides a method for treating arthritis, Benign Prostatic Hyperplasia (BPH), prostititous, including prostititous of the female prostate, an infection of the genitourinary tract, or a hemorrhoidal disease in humans or animals, the method comprising administering the presently claimed compositions.

The present invention also provides a method of improving immunity or activating Vitamin D receptor in humans or animals, the method comprising administering the presently claimed compositions.

The present invention also provides a method of treating metabolic syndromes in humans or animals, the method comprising administering the presently claimed compositions.

### DETAILED DESCRIPTION

Illustrative, non-limiting embodiments of inventions described herein are set forth illustratively in the following numbered paragraphs in this section. The phrase “In accordance with any of the foregoing or the following” used in these numbered paragraphs refers to the other numbered paragraphs in the section. In these paragraphs the phrase explicitly means that the illustrative embodiments described herein include the subject matter set forth in each numbered paragraph by itself and in any combination with the subject matter of any one or more of the other numbered paragraphs. It is explicitly applicant's purpose in setting forth these numbered paragraphs to describe various embodiments by the paragraphs taken alone and in any combination with one another, including, where any paragraph lists alternatives, any one or more of the alternatives in combination with any one or more of the alternatives listed in any one or more of the other paragraphs. Embodiments are thus commodiously disclosed as including the subject matter of each paragraph alone, including alternatives, and in any combination with the subject matter of any other paragraphs, including any one or more of alternatives therein set forth. Applicant specifically reserves the right to claim any of the subject matter set out in these paragraphs, taking the paragraphs alone or in any combination, including any one or more alternatives listed in any one or more of the paragraphs taken alone or in any combination with one or more of any other paragraphs.

Definitions

IU (International Unit) is used herein in relationship to the various forms of Vitamin D and derivatives thereto to denote International Unit and, as described below, is used herein to denote 65 picomoles Vitamin D3 and equivalents thereof.

For Vitamin D3 as used herein refers to (13,5Z,7E)-9,10-secocholesta-5,7,10(19)-trien-3-ol, the IUPAC name for Vitamin D3, and also refers to cholecalciferol and activated 7-dehydrocholesterol., one IU of Vitamin D3 is also 65 picomoles of pure 313,5Z,7 E)-9,10-secocholesta-5,7,10(19)-trien-3-ol, where one IU is 0.025 micrograms, and the molecular weight of Vitamin D3 is 384.64 g/mol. It will be appreciated that the mass of 65 picomoles of a substance will depend on its molecular weight and is different for substances of different molecular weights.

As used herein, the “activity” in a given assay, such as an assay of Vitamin D activity, of a given amount of a substance, such as 65 picomoles, will depend on the specific activity of the substance, and will depend on the intrinsic specific activity of the substance if the substance is in completely pure and fully active form.

For purposes herein, should there be ambiguity or doubt about its meaning, one IU of a Vitamin D substance means 65 picomoles of the substance.

A gram equivalent of Amaranthus blitoides , as that term is used herein, is one gram of dried leaves of Amaranthus blitoides , or the amount of any extract, derivative, infusion, and the like derived or made from one gram of dried leaves. For instance, one gram equivalent of a liquid infusion of Amaranthus blitoides would be the amount of the infusion obtained per gram of dried leaves. For an infusion made by steeping 25 grams of dried leaves in 250 ml of water, 10 ml would contain a gram equivalent of the Amaranthus. If the same infusion was concentrated from 250 ml to 50 ml, each 2 ml would contain a gram equivalent of the Amaranthus.

The term container is used herein in this respect to mean much the same thing as pouch or bag or the like, particularly in reference the use of a water permeable barrier suitable for holding the compositions herein disclosed while at the same time allowing them to come in contact with liquid, infuse there into, and then be conveniently removed there from, in much the same manner as a tea bag serves as a container for tea allowing the tea to be put in contact with hot water, infuse it, and then be conveniently removed from the water.

Vitamin D compounds as referred to herein may include any commercially available formulations suitable for human consumption. These and other such formulations may be used in accordance with various embodiments of the inventions herein described.

Herein described are supplemented Amaranthus blitoides compositions useful for relief of symptoms and discomfort associated with inflammation, such as that caused by inflammatory disorders, Benign Prostate Hyperplasia, prostititous, including prostititous of the female prostate, infections of the genitourinary tract, hemorrhoidal diseases, and inflammatory disorders to name a few. The supplemented compositions as disclosed herein also are useful to treat the same, and methods are herein disclosed for using them to that end.

In an embodiment Amaranthus blitoides leaves are used. In an embodiment the leaves are dried. In an embodiment the leaves are any one or more of minced, chopped, crushed, pulverized, powdered, or broken up in other ways. In an embodiment that leaves can be prepared for use in or for making any one or more of infusions such as teas, tinctures, extracts such as concentrated extracts for making beverages including, for instance, health, nutritional supplement and sport drinks, and for formulation into, among others, solid dispersions, liquid dispersions, capsule, tablet or other forms.

The present invention adds Vitamin D to Amaranthus blitoides , especially vitamin D3. The composition in the present invention can effectively alleviate inflammation and can treat BPH, and has good application prospect. It can improve immunity, activate vitamin D receptor, treat inflammation, cure arthritis, Benign Prostatic Hyperplasia (BPH), prostititous, including prostititous of the female prostate, an infection of the genitourinary tract, a hemorrhoidal disease.

Extended Half-Life versus Fortification. Inflammation affects more than 50 percent of the world population and is the primary cause of many disease types such as inflammation of the Prostate gland and Rheumatoid Arthritis. In our study we noted that extending the Half- Life (making it last longer) of the inherent vitamin D-3 in Amranthus blitoides extends the effectiveness, viability and efficacy of the extract for treatment purposes. This Half-Life extension both increases shelf life and helps to ensure a consistent infusion of the extract to boost the activation of the VDR human response receptor that reduces swelling or inflammation and provides the necessary protection and holds inflammation at acceptable levels and controls the manifestation of increased inflammation and the spread of other inflammation related diseases. The active form of vitamin D binds to the VDR and regulates transcriptional responses in humans, in humans the vitamin receptor is encoded by the VDR gene.

A common misnomer deserving of further explanation is the widespread belief that fortifying or adding to medicine will extend the effectiveness. Unlike fortification with unrelated synthetic compounds, the present invention utilizes biofortification of Vitamin D and its derivatives with the Vitamin D found naturally in Amaranthus blitoides . This biofortification extends the half life of Vitamin D and its derivatives and ensures the continuous dosing to create positive and consistent outcomes in the treatment of all inflammation related disease types. This biofortification also has the advantage of no necessary artificial additives to fortify Vitamin D.

In the composition of the invention, Amaranthus blitoides belong to grass plants instead of grain. Amaranthus blitoides itself can resist pests and diseases, and does not need to spray pesticides. Therefore, the composition of the invention can be prepared into animal feed, which can overcome the various defects of the feed prepared from the existing grain. Such as pesticide residues, difficult to digest and so on.

Amaranthus blitoides-Vitamin D Compositions

Certain embodiments provide compositions comprising Amaranthus blitoides and Vitamin D. In certain embodiments the Amaranthus blitoides and Vitamin D are obtained and combined in conventional ways. In certain embodiments the Amaranthus blitoides and Vitamin D are obtained from commercial suppliers. In certain embodiments the Amaranthus blitoides and Vitamin Dare mixed together by conventional means. In certain embodiments Vitamin D is formulated for water solubility and the composition comprising Amaranthus blitoides and Vitamin D is suitable for making a beverage, such as a beverage for human consumption. In certain embodiments, the composition comprising Amaranthus blitoides and Vitamin D is suitable for making a tea, such as a tea suitable for human consumption. In certain embodiments the composition comprising Amaranthus blitoides and Vitamin D is disposed in a container for making an infusion or tea, such as a tea bag.

In certain embodiments the Amaranthus blitoides is Amaranthus blitoides S. Watson.

In certain embodiments the Vitamin D is one or more of Vitamin D1 , Vitamin D2 , Vitamin D3 , Vitamin D4 , Vitamin D5 or a derivative thereof effective to provide the benefits of Vitamin D to a subject.

In certain embodiments the Amaranthus blitoides is cut, minced, crushed, powdered or pulverized or a mixture of any one or more thereof.

In certain embodiments the Amaranthus blitoides is in the form of whole leaves, minced leaves, crushed leaves, powdered leaves, pulverized leaves or a mixture of any one or more of the foregoing in any proportion thereof.

In certain embodiment the ratio of Vitamin D to Amaranthus blitoides is any 25 of about 1, 2, 3, 4, 5, 10, 15, 20, 25, 30, 40, 50, 60, 75, 100, 125, 150, 175, 200, 225, 250, 275, 300, 325, or 350 IU of Vitamin D per gram equivalent of Amaranthus blitoides.

In certain embodiment the ratio of Vitamin D to Amaranthus blitoides is in the range of any of 1-2,000, 2-2,000, 5-1,000, 5-500, 10-1,000, 10-500, 20-1,000, 20-500, 50-1,000, 50-500, 50-250, 50-125, 25-250, 25-750, 75-750, 75-500, 75-225, 0.1-100, 0.5-50, 0.25-25, 1-25, 1-15, or 1-10 IU of Vitamin D per gram equivalent of Amaranthus blitoides.

In certain embodiments the composition further comprises substances, such as additional substances suitable for human or animal consumption.

In certain embodiments the composition is sugar-free and gluten-free.

In certain embodiments the composition further comprises any one or more of the following in any number and combination: fillers, preservatives, colorants, sweeteners, or flavorings suitable for human consumption.

In certain embodiments the composition further comprises any one or more of additional metals, minerals, organics, vitamins, amino acids and other substances or nutrients, such as those suitable for human consumption.

In certain embodiments the composition comprises, any one or more of the following: zinc, copper, iodine, chromium, iron, molybdenum, selenium, silicon, boron, vanadium, germanium, potassium, calcium and/or magnesium; polyphenolics, sterols, such as sitosterol, and/or free fatty acids; Vitamin C, Vitamin E, Vitamin B-6 and/or other vitamins; lysine, glycine and/or tyrosine; saw palmetto, lycopene, quercetin, Pyseum africanum, stinging nettles, pumpkin seed oil, sasparilla, ginseng, Bubellabroom, milk thistle, kelp, Uva ursi extract, flower pollen, bosvellio, flaxseed, bees wax, calcium D glycan, and/or glycinine, such as to all of the foregoing those suitable for human consumption in amounts beneficial to human health.

In certain embodiments the composition is an extract, tincture or other liquid made from Amaranthus blitoides supplemented with Vitamin D in the ratios set forth herein, and optionally further supplemented with any one or combination of ingredient set forth above, such as but not limited to thickeners, fillers, preservatives, colorants, sweeteners, or flavorings. In embodiments the extract is a concentrated extract for making a tea, sport drink or other beverage, such as by dilution.

In some embodiments the composition is a beverage, such as a beverage suitable for human consumption, such as, among others a tea, sport drink, energy drink, lemonade, juice or juice drink, soft drink, water or flavored water drink or nutritional food drink with the concentration of Amaranthus blitoides , Vitamin D and optionally other ingredients set forth above and elsewhere herein.

In some embodiments the composition is disposed in a capsule, caplet, tablet, tab or other solid formulation suitable for human use. In embodiments the composition is disposed in water permeable container, such as a container for brewing tea. In embodiments the composition is disposed in a container comprising any one or more of the following: filter paper, nylon, silk or Soilon.

Some embodiments provide a water permeable container having disposed therein a composition according to any of the foregoing or the following in an amount of about 0.1, 0.2, 0.5, 1.0, 2.0, 3.0, 4.0, 5.0, 7.0, 10, 15, 20, 25, 50, 100, 150, 200, 250 or 500 grams.

Some embodiments provide a water permeable container having disposed therein a composition according to any of the foregoing or the following in the range of any of 0.1-100, 0.5-500, 0.5-25, 1-5, 1-10, 1-10,000, 2-4, 2-20, 2-200, 15-150, 25-750 or 50-5,000 grams.

Some embodiments provide a caplet, tablet, tab or other solid formulation, such as those suitable for human use, comprising any of about 0.1, 0.2, 0.5, 1.0, 2.0, 3.0,4.0, 5.0, 7.0, 10, 15, 20, 25, 50, 100, 150, 200, 250 or 500 grams of a composition as described above and elsewhere herein.

Some embodiments provide a capsule, caplet, tablet, tab or other solid formulation suitable for human use comprising any of about 0.1 to 0.5, 0.2 to 1.0, 0.5 to 2.0, 1.0 to 3.0, 2.0 to 4.0, 3.0 to 5.0, 2.5 to 7.5 5.0 to 10, 7.5 to 15, 10 to 20, 15 to 30, 25 to 50, 30 to 60, 50 to 100, 75 to 125, 100 to 150, 125 to 175, 150 to 200, 175 to 250 200 to 300, or 250 to 500 or more grams of a composition as described above and elsewhere herein.

Methods of Treatment

Compositions comprising Amaranthus blitoides and Vitamin D as described herein may be used to reduce symptoms and/or discomfort associated with inflammation, arthritis, Benign Prostatic Hyperplasia (BPH), prostititous, including prostititous of the female prostate, an infection of the genitourinary tract, and/or a hemorrhoidal disease. In embodiments the treatment does not cause unwanted sexual dysfunction.

Compositions comprising Amaranthus blitoides and Vitamin D as described herein may be used to treat inflammation, arthritis, Benign Prostatic Hyperplasia (BPH), prostititous, including prostititous of the female prostate, an infection of the genitourinary tract, and/or a hemorrhoidal disease. In embodiments treatment does not cause unwanted sexual dysfunction.

Embodiments provide methods for treating one or more symptoms and/or discomforts due to any one or more of inflammation, arthritis, Benign Prostatic Hyperplasia (BPH), prostititous, including prostititous of the female prostate, an infection of the genitourinary tract, and/or a hemorrhoidal disease, comprising administering to a subject in need thereof an Amaranthus blitoides-Vitamin D composition as described above and elsewhere herein.

Some embodiments provide methods for treating one or more symptoms and/or discomforts due to an immune-related disorder, comprising administering to a subject in need thereof an Amaranthus blitoides-Vitamin D composition as described above and elsewhere herein.

Some embodiments provide methods for treating any one or more of inflammation, Benign Prostatic Hyperplasia (BPH), prostititous, including prostititous of the female prostate, infections of the genitourinary tract, and/or a hemorrhoidal diseases, comprising administering an Amaranthus blitoides-Vitamin D composition as described above and elsewhere herein.

Some embodiments provide methods for treating an immune-related disorder, comprising administering an Amaranthus blitoides-Vitamin D composition as described above and elsewhere herein.

In the present specification, all disclosed features or all disclosed methods or steps in the process can be combined in any way except for mutually exclusive features and/or steps.

In the present specification, any disclosed feature can be replaced by other equivalent or similar alternative features, unless particular statement. That is, unless particular statement, each feature only is an example of a series of equivalent or similar features.

### EXAMPLES

The following Examples are provided by way of illustration only and are in no way limiting of the inventions herein described or their embodiments. A more complete understanding of the inventions herein described is to be had only by reading the present application in its entirety as it would be understood by those persons skilled in the arts to which it pertains.

### Example 1

### Preparation of Amaranthus blitoides

Amaranthus blitoides leaves are removed from the plant and allowed to dry. The leaves are then crushed or chopped.

### Example 2

### Preparation of Amaranthus blitoides-Vitamin D Compositions

Two kilograms of crushed dried leaves of Amaranthus blitoides mixed with one gram of CapsuDar® D3 100E (100,000 IU/g), a microencapsulated powder formulation of Vitamin D3 for foods, providing a composition-Vitamin D composition with 100 IU (1 milligram) Vitamin D3/2 grams Amaranthus blitoides.

### Example 3

### Preparation of Tea Bags of Enriched Maranthus blitoides

Two grams each of the composition set out in Examiner 2 are introduced into tea bags in accordance with standard practices for making tea bags use in foods for human consumption.

Each tea bag thus produced contains approximately 2 grams dried leaves of Amaranthus blitoides and 1 milligram of microencapsulated Vitamin D preparation.

Fully extracted the Vitamin D3 in each tea bag provides 100 IU, about the same as a cup of Vitamin D3 fortified milk sold in the United States.

### Example 4

### Amaranthus blitoides-Vitamin D tea

A tea bag containing Amaranthus blitoides enriched with Vitamin D according to Example 3 is steeped in one cup of boiled water for approximately 5 minutes. The resulting solution is enriched tea in accordance an embodiment of the invention herein described.

### Example 5

### Concentrate of Sport Drink

Amaranthus blitoides tea is brewed as described in Example 4. The tea is concentrated 100 fold by vacuum evaporation or freeze evaporation (lyophilization). The resulting concentrate is added to a sport drink formulation in a ratio of 1:99 to provide Amaranthus blitoides-Vitamin D3 enriched sport drink. Alternatively the concentrate is made from Amaranthus blitoides prior to enrichment with Vitamin D3, and Vitamin D3 is added in the appropriate amount to the concentrate, to the sport drink formulation or to the sport drink.

### Example 6

### Capsules

For the production of a capsule, dried pulverized leaves of Amaranthus blitoides is mixed with Vitamin D3 powder and put into a pharmaceutically acceptable capsule.

### Example 7

### Preparation of Tea Bags of Enriched Maranthus blitoides

The crushed dried leaves of Amaranthus blitoides in Example 1 and Vitamin D are introduced into tea bags in accordance with standard practices for making tea bags use in foods for human consumption.

Each tea bag thus produced contains approximately 5 grams dried leaves of Amaranthus blitoides and 0.012 milligram (400 IUs) of Vitamin D3.

### Example 8

### Treatment of Symptoms Associated with Inflammation

A cup of tea prepared as described in Example 4 is ingested twice to quintic each day for at least two weeks by one hundred subjects suffering from symptoms associated with inflammation, and the tea volume equals 8 ounces each time.

By the end of the two weeks the subjects experience at least partial relief from the symptoms.

### Example 9

### Treatment of Symptoms Associated with Inflammation

Two capsules of A. blitoides-Vitamin D3 as described in Example 6 is ingested triple to quintic each day for at least two weeks by one hundred subjects suffering from symptoms associated with inflammation.

By the end of the two weeks the subjects experience at least partial relief from the symptoms.

### Example 10

### Treatment of Symptoms Associated with BPH

A cup of tea prepared as described in Example 4 is ingested twice to quintic each day for at least two weeks by one hundred subjects suffering from symptoms associated with BPH, and the tea volume equals 8 ounces each time.

By the end of the two weeks the subjects experience at least partial relief from the symptoms, especially inflammation.

### Example 11

### Treatment of Symptoms Associated with BPH

The two capsules capsule of A. blitoides-Vitamin D3 as described in Example 6 is ingested triple to quintic each day for at least two weeks by one hundred subjects suffering from symptoms associated with BPH.

By the end of two weeks the subjects experience at least partial relief from the symptoms of BPH, especially inflammation.

1. A composition having enhanced Vitamin D half life comprising Amaranthus blitoides and Vitamin D or its derivatives.

2. The composition of claim 1, wherein the half life of Vitamin D or its derivatives is extended.

3. The composition of claim 1, wherein the Vitamin D or its derivatives is substantially one or more of Vitamin D1, Vitamin D2, Vitamin D3, Vitamin D4, Vitamin D5, or one or more derivatives thereof.

4. The composition according to claim 1, wherein the Amaranthus blitoides is Amaranthus blitoides Watson.

5. The composition of claim 1, wherein the Amaranthus blitoides comprises dried leaves of Amaranthus blitoides Watson.

6. The composition of claim 5, wherein the dried leaves comprise whole leaves, fragments, powder, or a mixture of whole leaves, fragments, and powder.

7. The composition of claim 1, wherein the ratio of Amaranthus blitoides to Vitamin D or its derivatives is 1 g to 1, 2, 3, 4, 5, 10, 15, 20, 25, 30, 40, 50, 60, 75, 100, 125, 150, 175, 200, 225, 250, 275, 300, 325 or 350 IU.

8. The composition of claim 1, wherein the ratio of Amaranthus blitoides to Vitamin D or its derivatives is 1 g to 1-2000 IU, 2 g to 100 IU, or 5 g to 400 IU.

9. The composition of claim 1, wherein the composition comprises minerals, organics, vitamins, amino acids, or other nutrients suitable for human consumption.

10. The composition of claim 9, wherein the composition comprises any one or more of the following: zinc, copper, iodine, chromium, iron, molybdenum, selenium, silicon, boron, vanadium, germanium, potassium, calcium and/or magnesium; polyphenolics, sterols, such as sitosterol, and/or free fatty acids; Vitamin C, Vitamin E, Vitamin B-6 and/or other vitamins; lysine, glycine and/or tyrosine; saw palmetto, lycopene, quercetin, Pyseum africanum, stinging netttles, pumpkin seed oil, sasparilla, ginseng, Bubella broom, milk thistle, kelp, Uva ursi extract, flower pollen, bosvellio, flaxseed, bees wax, calcium D glycan, and/or glycinine.

11. The composition of claim 1, wherein the composition comprises animal feed.

12. The composition of claim 1, wherein the composition comprises a food and one or more pharmaceutically acceptable excipients or auxiliary constituents.

13. The composition of claim 12, wherein the food comprises candy, bread, cake, pastry, or biscuits.

14. The composition of claim 12, wherein the food comprises a liquid.

15. The composition of claim 14, wherein the composition is placed in a water permeable container.

16. The composition of claim 15, wherein the water permeable container comprises filter paper, nylon, silk or soilon, or tea bags.

17. The composition of claim 15, wherein the ratio of Amaranthus blitoides to Vitamin D is 2 g to 100 IU or 5 g to 400 IU.

18. The composition of claim 15, wherein in one water permeable container, the weight of Amaranthus blitoides is 0.1 to 500 g.

19. The composition of claim 14, wherein the liquid comprises an energy drink, tea, coffee, lemonade, juice, juice drink, soft drink, water, flavored water drink, or nutritional food drink.

20. The composition of claim 12, wherein the food is tea, tablet, capsule, oral liquid, pulvis, decoction, granule, or pill.

21. The composition of claim 20, wherein the ratio of Amaranthus blitoides to Vitamin D is 0.6718 g to 400 IU.

22. The composition of claim 20, wherein the weight of Amaranthus blitoides comprises 0.6718 g, and the Vitamin D comprises 400 IU.

23. The composition of claim 20, wherein the weight of Amaranthus blitoides is 0.1 to 500 g.

24. A composition having enhanced Vitamin D half life comprising Amaranthus blitoides and Vitamin D or its derivatives, wherein the half life of Vitamin D or its derivatives is extended, wherein the Vitamin D or its derivatives is substantially one or more of Vitamin D1, Vitamin D2, Vitamin D3, Vitamin D4, Vitamin D5 or one or more derivatives thereof, wherein the Amaranthus blitoides is Amaranthus blitoides Watson, and wherein the ratio of Amaranthus blitoides to Vitamin D or its derivatives is 1 g to 1-2000 IU, 2 g to 100 IU, or 5 g to 400 IU.

25. The composition of claim 24, wherein the composition comprises any one or more of the following: zinc, copper, iodine, chromium, iron, molybdenum, selenium, silicon, boron, vanadium, germanium, potassium, calcium and/or magnesium; polyphenolics, sterols, such as sitosterol, and/or free fatty acids; Vitamin C, Vitamin E, Vitamin B-6 and/or other vitamins; lysine, glycine and/or tyrosine; saw palmetto, lycopene, quercetin, Pyseum africanum, stinging netttles, pumpkin seed oil, sasparilla, ginseng, Bubella broom, milk thistle, kelp, Uva ursi extract, flower pollen, bosvellio, flaxseed, bees wax, calcium D glycan, and/or glycinine.

26. The composition of claim 24, wherein the composition comprises a food and one or more pharmaceutically acceptable excipients or auxiliary constituents.

27. The composition of claim 26, wherein the food comprises a liquid, wherein the composition is placed in a water permeable container, and wherein in one water permeable container the weight of Amaranthus blitoides is 0.1 to 500 g.

28. The composition of claim 26, wherein the food comprises tea, tablet, capsule, oral liquid, pulvis, decoction, granule, or pill, and wherein the weight of Amaranthus blitoides is 0.1 to 500.

29. A method for treating inflammation in humans or animals, the method comprising administering the composition of claim 1.

30. A method for treating arthritis, Benign Prostatic Hyperplasia (BPH), prostititous, including prostititous of the female prostate, an infection of the genitourinary tract, or a hemorrhoidal disease in humans or animals, the method comprising administering the composition of claim 1.

31. A method of improving immunity or activating Vitamin D receptor in humans or animals, the method comprising administering the composition of claim 1.

32. A method of treating metabolic syndromes in humans or animals, the method comprising administering the composition of claim 1.
